(19) United States
(12) Patent Application Publication
(10) Pub. No.: US 2025/0186457 A1
(43) Pub. Date: Jun. 12, 2025

(54) USE OF AN ORGANOMETALLIC RUTHENIUM COMPOUND FOR THE PREVENTION AND/OR TREATMENT OF DISEASES AND/OR CONDITIONS RELATED TO CANCER IN AN INDIVIDUAL

(71) Applicants: FACULDADE DE CIÊNCIAS DA UNIVERSIDADE DE LISBOA, Lisboa (PT); UNIVERSIDADE DO MINHO, Braga (PT)

(72) Inventors: Andreia MARQUES VALENTE, Lisboa (PT); Maria Helena ANSELMO VIEGAS GARCIA, Lisboa (PT); Ana Arminda LOPES PRETO DE ALMEIDA, Porto (PT); Ana Rita PINTO BRÁS, Pinheiro, Guimarães (PT)

(21) Appl. No.: 18/837,191

(22) PCT Filed: Feb. 20, 2023
(86) PCT No.: PCT/IB2023/051527

§ 371 Date: Aug. 9, 2024

(30) Foreign Application Priority Data
Feb. 22, 2022 (PT) 117807

(51) Int. Cl. A61K 31/555; A61P 35/00

(52) U.S. Cl. CPC A61K 31/555; A61P 35/00

(57) ABSTRACT

The present invention relates to an organometallic ruthenium compound of general formula (I) for use in the prevention and/or treatment of diseases and/or conditions related to colorectal cancer, pancreatic ductal adenocarcinoma, non-small cell lung cancer, and ovarian and endometrial cancer in an individual:

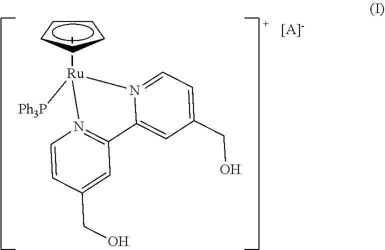

wherein:
A is selected from the group consisting of CF₃SO₃, PF₆, Cl, BPh₄, NO₃ or BF₄; said organometallic ruthenium compound of formula (I) being in all the possible racemic, and stereoisomeric forms.

### TECHNICAL FIELD

The present invention refers to an organometallic ruthenium compound for its use in the prevention and/or treatment of diseases and/or conditions related to cancer in an individual, more specifically, colorectal cancer, pancreatic ductal adenocarcinoma, non-small cell lung cancer, ovarian and endometrial cancer.

### BACKGROUND ART

Colorectal cancer (CRC) is an important cause of global morbidity and mortality, being the second leading cause of cancer death. In 2020, were estimated 1.1 million cases and more than 500,000 deaths worldwide (Sung et al., 2021).

Several oncogenes are involved in CRC carcinogenesis, being the most common mutations found in KRAS (40%), PIK3CA (20%) and BRAF (15%) (László et al., 2021; Laurent-Puig et al., 2009). These mutations activate key signaling pathways involved in the progression of CRC, namely MAPK and PI3K signaling pathways, that are responsible for fundamental cellular processes such as proliferation, apoptosis, autophagy, and invasion/motility (Berg & Soreide, 2012).

KRAS is the most frequently mutated oncogene in cancer and KRAS mutation is commonly associated with poor prognosis and resistance to therapy. More specifically, KRAS mutations predominate in cancers such as CRC, pancreatic ductal adenocarcinoma (PDAC), and non-small cell lung cancer (NSCLC) (Haigis, 2017).

Also, although KRAS can be mutated at a low percentage in almost any cancer types, higher than 10% mutation rates characterizes only ovarian and endometrial cancers (Timar & Kashofer, 2020).

The choice of treatment method for cancer is very important because each cancer responds to different methods differentially. Currently, four approaches are used, namely surgery, chemotherapy, radiotherapy and targeted therapy.

For CRC, for example, surgery is the mainstay treatment, followed by chemotherapy with 5-fluorouracil (5-FU). 5-FU is the most widely used agent to treat CRC and can be used alone or in combination with other drugs. However, it possesses success rates as low as 10%-15% due to severe side effects and resistance (Cazzanelli et al., 2016; Pardini et al., 2011; Van Cutsem et al., 2016).

Platinum anticancer agents are the group of anticancer drugs with the greatest success stories in the field of medicinal inorganic chemistry. Cisplatin was the first to be discovered and is the most used agent of the set. Despite the clinical success of cisplatin, it has many disadvantages associated. Chemotherapy with cisplatin is frequently accompanied by severe side effects and its activity is limited to a small spectrum of tumors due to acquired and intrinsic resistance to treatments (Bergamo et al., 2012).

One of the first cancer types where target therapy (anti-EGFR and anti-VEGFR) was introduced was CRC and testing for RAS mutation became a routine molecular pathology activity leading to several studies on KRAS clonality and primary/metastasis comparisons. The compounds bevacizumab, cetuximab, and panitumumab, for example, are usually used to target EGFR and VEGFR, respectively (Lee & Sun, 2016; Zaniboni, 2015). These receptors activate MAPK and PI3K signaling pathways, the most important signaling pathways in CRC carcinogenesis. Although, recent works have demonstrated that tumors with mutations in key downstream effectors of EGFR signaling pathways, as KRAS, BRAF, PIK3CA, do not correspond to EGFR antibodies, what creates a clinically relevant problem that needs to be overcome (Hong et al., 2016; Temraz et al., 2015).

Because of the severe side effects of the available therapies and acquisition of resistance of some tumors, research has been directed toward the development of compounds based on other metals. Among the several metal complexes explored so far, ruthenium (hereafter also referred by its chemical symbol—Ru) compounds appear as some of the most promising metallodrugs. Among other characteristics, Ru-based agents have been showing good anticancer activity, different modes of action, chemical stability, structural variety, with diverse ligand bonding modes and redox properties achievable, being some complexes quite selective for cancer cells (Moreira et al., 2019; Thota et al., 2018). Currently, there are some ruthenium complexes in clinical trials for use in cancer therapy (Alsaab et al., 2020; Thota et al., 2018).

The first goal of researchers working in therapeutic approaches to treat cancer is to try to discover new anticancer drugs with higher efficacy, reduced toxicity, lack of cross-resistance and improved pharmacological characteristics.

Despite recent progresses in cancer therapy, much remains to be done regarding available drugs and their side effects. In the case of CRC, therapies remain limited and patients with CRC that harbor mutations in KRAS do not respond to the available treatments (Hong et al., 2016).

Thus, the present invention proposes a new and inventive organometallic ruthenium compound for use in the prevention and/or treatment of diseases and/or conditions related to cancer in an individual, more specifically, colorectal cancer, pancreatic ductal adenocarcinoma, non-small cell lung cancer, ovarian and endometrial cancer.

### SUMMARY OF INVENTION

The present invention relates to an organometallic ruthenium compound having the general formula (I):

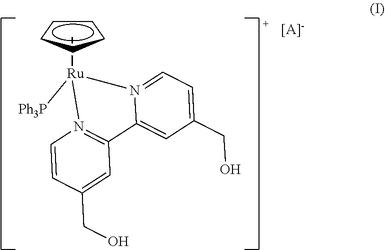

wherein:
A is selected from the group consisting of CF₃SO₃, PF₆, Cl, BPh₄, NO₃ or BF₄;
said organometallic ruthenium compound of formula (I) being in all the possible racemic, and stereoisomeric forms;
for use in the prevention and/or treatment of diseases and/or conditions related to colorectal cancer, pancreatic ductal adenocarcinoma, non-small cell lung cancer, and ovarian and endometrial cancer in an individual.

### Technical Problem

The lack of specific anticancer agents for targeting KRAS in cancer harboring this mutation highlights the need of developing new target-drugs. This problem is a major challenge and a relevant clinical concern that needs to be solved in cancer therapy.

Along the years, several strategies attempted to indirectly target KRAS through three distinct modes. The first one to be studied was the targeting of KRAS membrane localization, inhibiting farnesyltransferase by blocking KRAS post-translational modification. However, these approaches do not discriminate between mutant and wild-type KRAS proteins and can affect a multitude of other proteins that undergo prenylation and post-translational processing by the same enzymes (Ryan & Corcoran, 2018a).

Another direction for the treatment of KRAS-driven tumors was the targeting of RAS GTP/GDP cycle using inhibitors for guanine nucleotide exchange factors such as Sos1, although the binding activity of these inhibitors to KRAS was weak and these inhibitors have not translated into clinical use (Nagasaka et al., 2020).

The last approach was the targeting of KRAS downstream effectors, using inhibitors against BRAF, MEK, ERK, AKT or mTOR proteins. However, the significant crosstalk between MAPK and PI3K signaling pathways and the drug resistance associated led to limited clinical benefits (Liu et al., 2021).

KRAS mutations are found in 40% of CRCs and alterations in codons 12 and 13 are the most frequent. The most common amino acid substitutions in CRC with KRAS mutations are G12D (13%), G12V (9%), G13D (7%) (László et al., 2021; Li et al., 2020). Also, KRAS mutations are present in 80% of PDAC, 35% of NSCLC and 10% of ovarian and endometrial cancer, with NRAS-another important member of the RAS family-being the second most frequently mutated (Christensen et al., 2020; Haigis, 2017; Timar & Kashofer, 2020).

Selectively targeting specific mutations in KRAS could provide a more direct approach to inhibit oncogenic KRAS mutant function while sparing the function of the wild-type protein.

In the last years, two KRAS^{G12C} inhibitors, sotorasib and adagrasib, have been accepted for clinical use in non-small cell lung cancer (NSCLC) (Briere et al., 2021; Canon et al., 2019; Fell et al., 2020; Hallin et al., 2020; Lanman et al., 2020; Skoulidis et al., 2021; Liu et al., 2021). However, KRAS^{G12C} mutation is only present in 3% of CRCs with KRAS mutation and these inhibitors are not effective in CRC with mutated KRAS (László et al., 2021).

Recent studies have identified a potent and selective KRAS^{G12D} inhibitor which antitumor benefit was demonstrated in a murine animal model (Xiaolun Wang et al., 2022; Kemp S B, 2023). Having achieved specific, potent, and durable effects in promoting tumor regression in the implantable and autochthonous pancreatic cancer models, such results support the potential for the development of an effective therapeutic tool.

In the last few years, more KRAS inhibitors entered in clinical trials, but they all target KRAS^{G12C} mutations (Janes et al., 2018; Molina-Arcas et al., 2019; Ostrem et al., 2013; Patricelli et al., 2016; Liu et al., 2021). To the best of our knowledge no KRAS inhibitors are available to specifically target KRAS hotspot mutations in CRC.

### Solution to Problem

Surprisingly, the present invention solves the problems of prior art by identifying an organometallic ruthenium compound having the formula (I) for use in the prevention and/or treatment of diseases and/or conditions related to cancer in an individual more specifically, colorectal cancer, pancreatic ductal adenocarcinoma, non-small cell lung cancer, ovarian and endometrial cancer.

Over the last few years, the inventors have been dedicated to a family of “piano stool” compounds (Ru(II)-cyclopentadienyl complexes) as potential anticancer drugs for chemotherapy (Valente et al., 2021).

Until now, the anticancer effects of a specific organometallic ruthenium compound having the formula (I), namely PMC79, were only evaluated in the ovarian cancer cell line, A2780, and breast cancer-derived cell lines, MCF7 and MDA-MB-231 (Moreira et al., 2019). The anticancer effects of this compound proved to be promising, with lower half-maximal inhibitory concentration (IC₅₀) for both models (3.9 UM, 5.9 UM and 2.1 uM for A2780, MCF7 and MDA-MB-231, respectively) and in the case of breast cancer cells with IC₅₀ values lower than cisplatin (36 UM and 110 UM for MCF7 and MDA-MB-231, respectively). The PMC79 compound is identified as ({2,2′-bipyridine}-4,4′-diyldimethanol-k²N, N′)(η⁵-cyclopentadienyl) (6riphenylphosphine) ruthenium (II) triflate.

The results also revealed that in the breast cancer cell lines, PMC79 inhibited proliferation by decreasing colony formation ability and induced apoptosis, demonstrated by increasing in Annexin V stained cells. PMC79 also induced alterations in mitochondria morphology, with edematous and disorganized mitochondria, and in actin cytoskeleton with cytoskeleton disorganization, cell dispersion and cell to cell cytoskeleton extensions reduction. Moreover, the results also revealed that PMC79 was preferentially distributed in membrane fraction (Moreira et al., 2019).

Some Ru(II)-cyclopentadienyl complexes of our group have already been tested in CRC cell lines but never PMC79 (Morais et al., 2016; Valente et al., 2021). Results show that even very small structural differences result in drastic biological responses (Morais et al., 2016; Valente et al., 2021). Also, the same ruthenium compound can show high cytotoxicity or not being cytotoxic at all depending on the cells genetic background (Teixeira et al., 2021). Thus, taking into consideration that PMC79 has been only tested in ovarian and breast cancer models and due to the genetic differences and distinct therapy approaches to these cancers, it would not be expected that this same compound would have application in CRC.

As mentioned above, KRAS mutations are the most frequent oncogenic alterations present in CRC (40%) as well as in PDAC (80%), in NSCLC (35%) and in ovarian and endometrial cancer (10%) (Christensen et al., 2020; Haigis, 2017; Timar & Kashofer, 2020). Therefore, in addition to CRC, it is also expected that this compound could have application in PDAC, NSCLC and ovarian and endometrial cancer due to the high incidence of KRAS mutations in these cancer models.

### BRIEF DESCRIPTION OF DRAWINGS

With the purpose of providing an understanding of the principles according to the embodiments of the present invention, reference will be made to the embodiments illustrated in the figures and to the terminology used to describe them. In any case, it must be understood that there is no intention of limiting the scope of the present invention to the content of the figures. Any subsequent alterations or modifications of the inventive characteristics shown herein, as well as any additional applications of the principles and embodiments of the invention shown, which would occur normally to a person skilled in the art having this description in hands, are considered as being within the scope of the claimed invention.

FIG. 1 illustrates the colony formation assay results, in SW480 colorectal cancer cell line, after incubation with a compound according to the present invention;

FIG. 2 illustrates the cell cycle results, in SW480 colorectal cancer cell line, after incubation with a compound according to the present invention;

FIG. 3 illustrates the CFSE assay results, in SW480 cells, after incubation with a compound according to the present invention;

FIG. 4 illustrates the Annexin V/Propidium iodide results, in SW480 colorectal cancer cell line, after incubation with a compound according to the present invention;

FIG. 5 illustrates the Terminal transferase dUTP nick end labeling (TUNEL) assay results, in SW480 cells, after incubation with a compound according to the present invention;

FIG. 6 illustrates the wound healing results, in SW480 cells, after incubation with a compound according to the present invention;

FIG. 7 illustrates an analysis of cellular distribution, in SW480 cells, after incubation with a compound according to the present invention;

FIG. 8 illustrates an analysis of F-actin staining in order to evaluate how the cytoskeleton of SW480 cancer cells is affected by a compound according to the present invention;

FIG. 9 illustrates a Western blot analysis of SW480 cell line considering the evaluation of the expression referred to KRAS, pAKT, total-AKT, PERK and total-ERK proteins induced by a compound according to the present invention;

FIG. 10 illustrates a Western blot analysis of RKO cell line considering the evaluation of the expression referred to KRAS, pAKT, total-AKT, PERK and total-ERK proteins induced by a compound according to the present invention;

FIG. 11 illustrates a Western blot analysis of LS174T cell line considering the evaluation of the expression referred to KRAS, pAKT, total-AKT, PERK and total-ERK proteins induced by a compound according to the present invention;

FIG. 12 illustrates a Western blot analysis of SW480 cell line considering the evaluation of the expression referred to KRAS protein induced by siRNA targeting KRAS and a compound according to the present invention.

### DESCRIPTION OF EMBODIMENTS

The present invention refers, in a first aspect, to an organometallic ruthenium compound having the general formula (I):

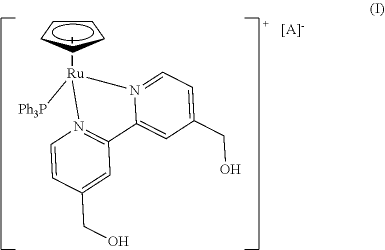

wherein:
A is selected from the group consisting of CF₃SO₃, PF₆, Cl, BPh₄, NO₃ or BF₄;
said organometallic ruthenium compound of formula (I) being in all the possible racemic, and stereoisomeric forms;
for use in the prevention and/or treatment of diseases and/or conditions related to colorectal cancer, pancreatic ductal adenocarcinoma, non-small cell lung cancer, and ovarian and endometrial cancer in an individual.

In the preferred embodiments according to the first aspect of the invention, the organometallic conjugate of formula (I) is:
({2,2′-bipyridine}-4,4′-diyldimethanol-k²N, N′)(η⁵-cyclopentadienyl) (9riphenylphosphine) ruthenium (II) triflate (compound PMC79).

In the preferred embodiments according to the invention, the individual is a mammal, in particular a human.

In other embodiments, the organometallic ruthenium compound for use is in a composition comprising a pharmaceutically acceptable excipient. The term “pharmaceutically acceptable” means what is useful in preparing a pharmaceutical composition generally safe, non-toxic, and neither biologically nor otherwise undesirable and includes what is acceptable for veterinary as well as human pharmaceutical use.

In other embodiments, the organometallic ruthenium compound for use is in a composition administered by oral, rectal or parenteral injection route, preferably by parenteral route. The term “parenteral injection” refers to an administration via injection under or through one or more layers of skin or mucus membranes of an individual. More preferably, said parenteral administration is selected from the group consisting of intravenous administration, intramuscular administration, intradermal administration, or subcutaneous administration.

In other embodiments, the organometallic ruthenium compound for use is in a composition in the form of tablets, pills, powders, lozenges, sachets, cachets, elixirs, suspensions, emulsions, solutions, syrups, aerosols, sprays, gels, soft and hard gelatin capsules, suppositories, sterile injectable solutions or sterile packages powders.

The organometallic ruthenium compound for use according to present invention inhibit KRAS and downstream signaling pathways MAPK and PI3K. The inhibition of KRAS and downstream signaling pathways MAPK and PI3K preferably results in inhibition of cellular proliferation and induction of apoptosis.

In a preferred embodiment of the present invention, the KRAS inhibitor target KRAS^{G12V} and KRAS^{G12D} mutations.

### Examples and Experimental Methods

### PMC79 Synthesis

The compound was synthesized using a protocol developed by us (Côrte-Real, Karas, Gírio, et al., 2019). Alternatively, the synthesis can be performed in methanol instead of dichloromethane.

### Cell Lines and Culture Conditions

The noncancerous NCM460 cell line derived from normal colon epithelial mucosa, was obtained from INCELL's (Moyer et al., 1996), and the colorectal cancer derived cell lines, SW480, LS174T and RKO, were obtained from American Type Culture Collection (ATCC). All cell lines were maintained at 37° C. under a humidified atmosphere containing 5% CO₂. NCM460 and SW480 cells were grown in RPMI medium and LS174T and RKO cells in DMEM, all supplemented with 10% FBS and 1% penicillin/streptomycin. SW480 cell were seeded normally at a concentration of 1×10⁵ cells/ml except for some specific assays.

### Sulforhodamine B Assay

CRC cells were seeded in 24-well test plates and after 24 h of seeding, cells were incubated with different concentrations of PMC79 for 48 h. Two negative controls were performed, a control (1) in which cells were incubated only with growth medium and a DMSO control (2) in which cells were exposed to the highest concentration used of DMSO (maximum of 0.1% DMSO per well (v/v))), to discard any influence of this solvent in the results. After 48 h of treatment, Sulforhodamine B was performed as previously described (Côrte-Real et al., 2019). Results were expressed relatively to the negative control (1), which was considered as 100% of cell growth.

### Colony Formation Assay

SW480 cell line was seeded, at a concentration of 500 cells/ml, in 6-well plates. 24 h after seeding, cells were incubated with ¼ IC₅₀ and ½ IC₅₀ values of PMC79. The negative control cells were treated with DMSO 0.1%. After 48h of incubation, the medium was replaced by fresh medium without PMC79. Medium was renewed every 3 days. Nine days after removing the treatments, cells were stained as previously described (Côrte-Real, Karas, Brás, et al., 2019).

### Carboxyfluorescein Succinimidyl Ester (CFSE) Assay

SW480 cells were incubated with the dye and seeded according to the procedure recommended by CFSE package insert (BD Horizon™). After 24h, cells were incubated with IC₅₀ and 2×IC₅₀ values of PMC79 and the cells of the 0 h time-point were collected and acquired on the flow cytometer. After 48 h and 72 h, both floating and attached cells were collected, washed with PBS, centrifuged at 500 rpm for 10 min and the pellet resuspended in PBS before cytometer acquisition.

### Cell Cycle Analysis

SW480 cell line was seeded in 6-well plates. After 24 h, cells were incubated with the IC₅₀ of PMC79. The negative control cells were treated with DMSO 0.1%. 24 h and 48 h later, cells were collected and processed according to the method described in (Teixeira et al., 2018). Cell-cycle phase data analysis and quantification was performed using FlowJo 10.7.2 software.

### Annexin V/Propidium Iodide Assay

SW480 was seeded in 6-well plates and 24 h after seeding, the cells were exposed to the IC₅₀ values of PMC79 compound. The negative control cells were treated with DMSO 0.1%. After 48 h, cells were collected and stained as previously described (Côrte-Real, Karas, Brás, et al., 2019). Acquisition was performed using CytoFLEX Cytometer (Beckman Coulter, Brea, CA, USA).

### TUNEL Assay

SW480 cell line was seeded, in 6-well plates and 24 h after seeding, cells were exposed to the IC₅₀ of PMC79. The negative control cells were treated with DMSO 0.1%. After 48 h, cells were processed according with (Teixeira et al., 2018). Slides were maintained at −20° C. until visualization in a fluorescence microscope (Leica DM 5000B, Leica Microsystems, Wetzlar, Germany).

### Wound Healing Assay

SW480 cell line was seeded at a concentration of 7×10⁵ cells/ml, in 6-well plates. After 24 h, a wound was made with a tip and cells were incubated with the IC₅₀ and 2×IC₅₀ of PMC79 for 12 h. The negative control cells were treated with DMSO 0.1%. Each condition was photographed at 0 h, 4 h, 8 h and 12 h. To analyze the data and measure the wound size at each time-point ImageJ 1.53a software was used.

### Cellular Distribution Measured by ICP-MS Analysis

SW480 cells were seeded into 100 mm petri dishes and after 24 h, cells were incubated with PMC79 at a concentration equivalent to its IC₅₀ value. After 48 h of incubation, cells were washed with ice-cold PBS and treated, to obtain a cellular pellet. The cytosol, membrane/particulate, cytoskeletal, and nuclear fractions were extracted using a Fraction-PREP (BioVision, USA) cell fractionation kit according to the manufacturer's protocol. The Ru (101Ru) content in each fraction was measured by a Thermo X-Series Quadrupole ICPMS (Thermo Scientific) after digestion of the samples and using the same procedure previously described (Côrte-Real et al., 2013).

F-Actin Staining with Phalloidin 568

SW480 cell line was seeded in 12-well plates with one coverslip per well. After 24 h, the cells were exposed to the IC₅₀ value of PMC79, and the cells of negative control were treated with DMSO 0.1%. After 48 hours of incubation, the cells were fixed and stained as previously described (Pilon et al., 2020). Representative images were obtained in a fluorescence microscope (Olympus motorized BX63F Upright Microscope) (Olympus@, Tokyo, Japan) at a magnification of 600×.

### Immunoblot Analysis

Preparation of total protein extracts of colorectal cancer cell lines (RKO, LS174T and SW480), SDS-PAGE and Western blots were performed as previously described (Alves et al., 2015).

### Antibodies

The Antibodies used were anti-KRAS and β-actin (Sigma); anti-phospho p44/42 MAPK (Thr202/Tyr204) (pERK), anti-p44/42 total (ERK), anti-phospho Akt (Ser473) (pAKT) and anti-Akt total (Cell Signaling) (AKT); GAPDH (Gene Tex). Proteomic study

SW480 cell line were seeded, in 100 mm Petri dishes and 24 h after seeding, cells were treated with PMC79 compound for protein extraction. Samples were processed for LC-MS/MS analysis (Chiva et al. 2018; Olivella et al. 2021). Protein identification and validation, and gene ontology, localization and network analysis were performed as previously described (Perkins et al., 1999; Beer et al., 2017; Perez-Riverol et al., 2022).

### Statistical Analysis

The results were obtained from at least three independent experiments and expressed as mean+SD. For analyzing the results were used a one-way ANOVA with Dunnett's post-test or Tukey's post-test and two-way ANOVA with Dunnett's post-test. All different statistical analyses were performed using GraphPad Prism version 8 for macOS, GraphPad Software, La Jolla California USA (http://www.graphpad.com).

### Examples and Results

### PMC79 has a Selective Anticancer Effect in Colorectal Cancer Cells

In order to evaluate the anticancer potential and determine the IC₅₀ of PMC79 compound, Sulforhodamine B assay was used.

PMC79 proved to inhibit cellular growth in SW480 CRC cell line, showing an IC₅₀ of 40 uM (Table 1), while the noncancerous cell line NCM460, shows a higher IC₅₀ value (44 uM).

Table 1 shows the IC₅₀ value and selectivity index determined at 48 h of incubation by Sulforhodamine B. The noncancerous NCM460 cell line derived from normal colon epithelial mucosa was used to determine the selectivity index for the colorectal cancer cell line, SW480. Values represent mean+SD of at least three independent experiments.

TABLE 1

| | IC₅₀ (μM) | | Selectivity Index |
|---|---|---|---|
| Compound | SW480^{KRAS} | NCM460 | NCM460/SW480 |
| PMC79 | 40.0 ± 2.0 | 44.0 ± 6.9 | 1.1 |

The selectivity index was also determined, demonstrating that PMC79 is selective to CRC cells (S.I.=1.1). Some authors support that a selectivity index higher than 1 indicates that cytotoxicity on cancer cells surpassed healthy non-tumoral ones (Ferreira et al., 2017). This result suggests that PMC79 is a good candidate to be explored as potential anticancer drug for CRC treatment.

### PMC79 Decreases Proliferation of Colorectal Cancer Cells

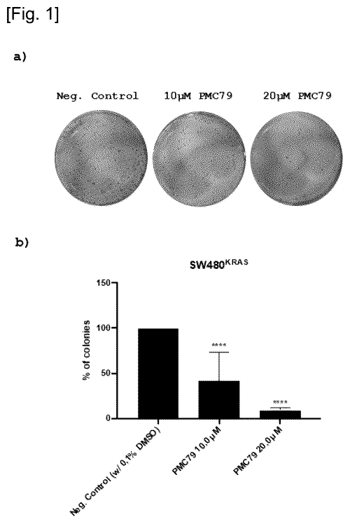

The effect of PMC79 on proliferation was studied using several techniques. It was performed a colony formation assay to evaluate the capacity of cells to recover and form colonies after a period of exposure to ¼ IC₅₀ and ½ IC₅₀ of PMC79. The results showed that PMC79 significantly decreased the clonogenic ability of SW480 cells in a dose-dependent manner (FIG. 1).

The FIG. 1 illustrates the analysis of the colony formation ability, after 48 h of incubation with ¼ IC₅₀ and ½ IC₅₀, in SW480 cell line. A) represents the images of colony formation assay in SW480 cell lines. B) shows the graphical representation of colony formation ability in SW480 cells. The values represent mean±S.D. of at least three independent experiments. Statistical analysis was performed by one-way ANOVA with Dunnett's multiple comparisons test. ****P≤ 0.0001 compared with negative control.

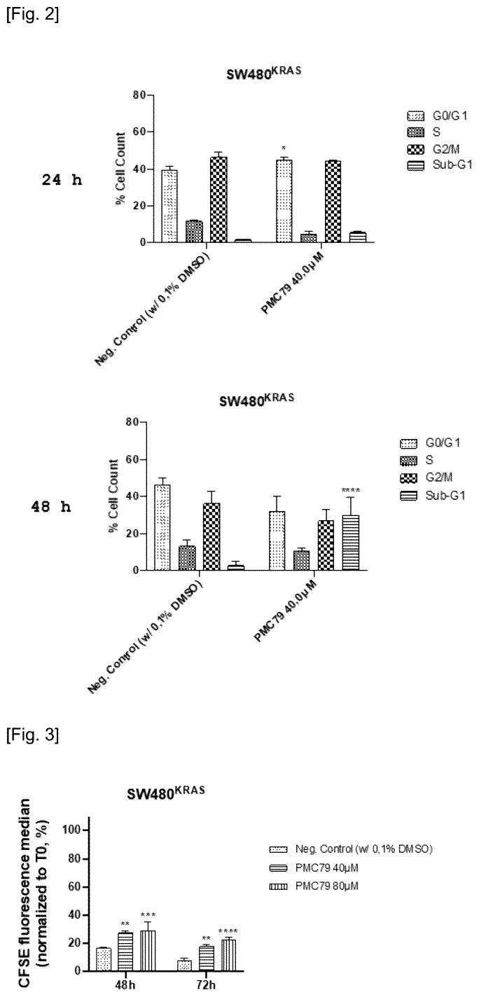

In order to assess the alterations induced by PMC79 in the cell cycle, SW480 cells were incubated with the IC₅₀ of PMC79 for 24 h and 48 h. It was noticed that at 24 h, PMC79 induced a cell cycle arrest at G0/G1 phase, however 24 h later an increase of sub-G1 population was observed, indicating that after 48 h PMC79 induces cell death (FIG. 2).

The FIG. 2 illustrates the cell cycle analysis using propidium iodide, after 24 h and 48 h of incubation with IC₅₀ of PMC79, in SW480 cell line. The values represent mean±S.D. of at least three independent experiments. Statistical analysis was performed by two-way ANOVA with Dunnett's multiple comparisons test. *P≤0.05; ****P≤0.0001 compared with negative control.

The effects on proliferation were also studied using carboxyfluorescein succinimidyl ester assay (CFSE). CFSE is a dye that allows monitoring cell division by flow cytometry, wherein the dye covalently binds to protein amine groups within the cells resulting in long lived fluorescent adducts. In each cell division, the fluorescence of CFSE-stained cells is reduced to half of the initial fluorescence. Thus, when cell proliferation is inhibited, we observe an increase of the intracellular fluorescence. SW480 cells were incubated with IC₅₀ and 2×IC₅₀ of PMC79 compound for 48 h and 72 h. The results supported the antiproliferative effect of PMC79 in CRC cells, showing a decrease in proliferation under all conditions (FIG. 3).

The FIG. 3 illustrates the quantification of CFSE median fluorescence intensity normalized to T0 after 48 and 72 h of incubation with IC₅₀ and 2×IC₅₀, in SW480 cell line. The values represent mean±S.D. of at least three independent experiments. Statistical analysis was performed by two-way ANOVA with Dunnett's multiple comparisons test. **P≤0.01; ***P≤0.001; ****P≤0.0001 compared with negative control.

### PMC79 Induces Apoptosis in Colorectal Cancer Cells

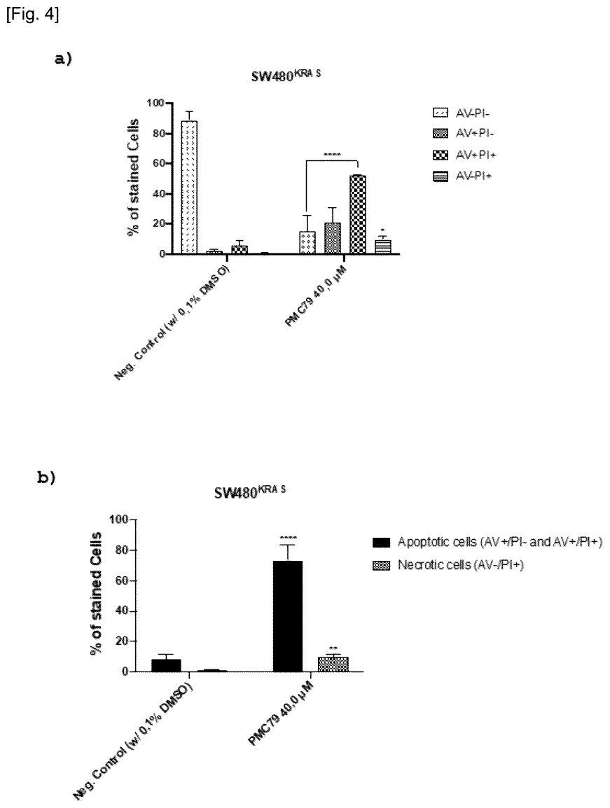

The effect of PMC79 compound on apoptosis was evaluated by flow cytometry using Annexin V/Propidium iodide (AV/PI) assay. CRC cells were incubated with IC₅₀ value, for 48 h and showed that the treatment with PMC79 led to a significant increase in the percentage of apoptotic cells (AV+/PI−; AV+/PI+) (74%) in comparison to the negative control (FIG. 4). Regarding the necrotic cells (AV−/PI+), it was also observed an increase in this type of cell death (10%), although in a less extent in comparison with the percentage of apoptotic cells.

The FIG. 4 illustrates the apoptotic cell death analysis. A) shows the graphical representation of Annexin V fluorescein isothiocyanate (AV-FITC) and propidium iodide (PI) assay in SW480 cells, after incubation with IC₅₀ value for 48 h. The values represent mean±S.D. of at least three independent experiments. Statistical analysis was performed by two-way ANOVA with Dunnett's multiple comparisons test. **P≤0.01; ****P≤0.0001 compared with negative control. B) shows the graphical representation of the total percentage of apoptotic (early apoptosis (AV+PI−) and late apoptosis (AV+PI+)) and necrotic cells (AV−PI+). Statistical analysis was performed by two-way ANOVA with Dunnett's multiple comparisons test. **P≤0.01; ****P≤0.0001 when compared with the negative control.

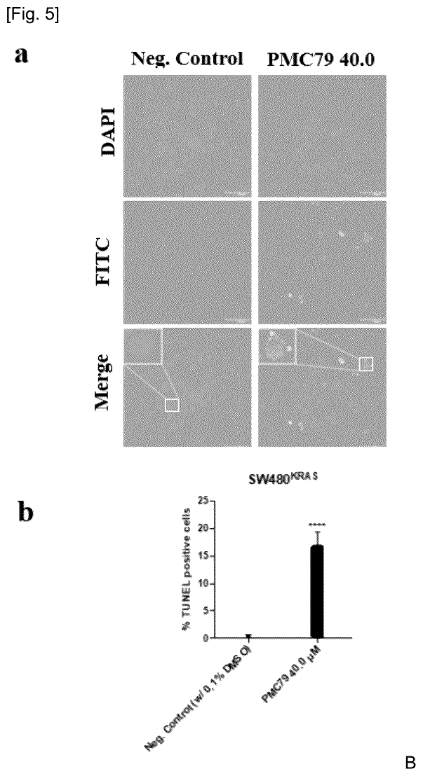

The levels of late apoptosis were also studied using TUNEL assay, after incubation of SW480 for 48 h with the IC₅₀ of PMC79. We observed an increase of the number of apoptotic cells (TUNEL positive) in PMC79 treated cells (FIG. 5). It was observed the presence of apoptotic bodies, phenotypic alterations typical of apoptosis (Velma et al., 2016) (FIG. 5a). This phenotypic alteration is one of the hallmarks of apoptosis confirming that the cell death induced by PMC79 is apoptosis.

The FIG. 5 illustrates the levels of TUNEL positive cells. SW480 cells were analyzed by TUNEL assay, after incubation with PMC79 IC₅₀ for 48 h. a) shows the representative images (×600) of TUNEL assay. DAPI (40,6-diamidino-2-phenylindole), FITC (fluorescein isothiocyanate) and merged were obtained by fluorescence microscopy. B) shows the analysis of TUNEL assay in SW480 cells. Values represent mean±S.D. of at least three independent experiments. Statistical analysis was performed by one-way ANOVA with Turkey's multiple comparisons test. ****P≤0.0001 compared with negative control.

These results are also in accordance with the data obtained in the cell cycle analysis showing an increase in percentage of cells in sub-G1 population after 48 h of incubation with PMC79. Overall, the results showed that cells exposed to PMC79 are mainly undergoing apoptosis.

### PMC79 Decreases Motility of Colorectal Cancer Cells

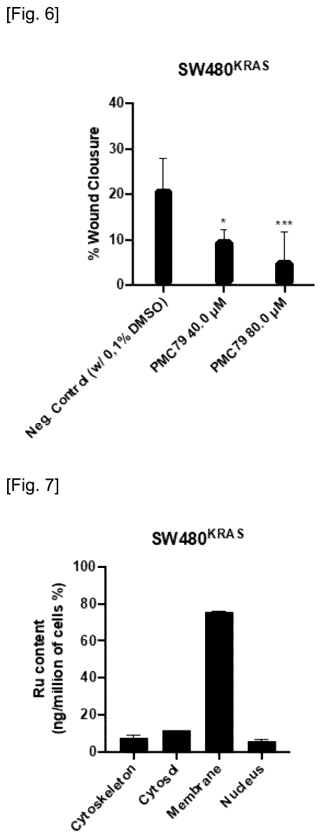

To determine the effect of PMC79 on cell migration, a wound healing assay was used. SW480 cells were incubated with IC₅₀ and 2×IC₅₀ of PMC79 and the wound size was measured every 4 h until completing 12 h of incubation. PMC79 decreased the percentage of wound closure in a dose-dependent manner, implying a reduction on cellular motility (FIG. 6). This result indicates that PMC79 decreases migration of SW480 cells.

The FIG. 6 illustrates the study of motility in SW480 cells using wound healing assay, after incubation with PMC79 IC₅₀ and 2×IC₅₀ for 12 h. Values represent mean±S.D. of at least three independent experiments. Statistical analysis was performed by one-way ANOVA with Turkey's multiple comparisons test. *P≤0.05; ***P≤0.001 compared with negative control.

### PMC79 is Preferentially Localized in the Membrane Fraction

The intracellular distribution of PMC79 was performed using a commercial kit as described in the experimental section. SW480 cells were incubated for 48 h at the IC₅₀ concentration. Cytosol, membrane, nucleus, and cytoskeletal fractions were extracted, and the result indicated that PMC79 is mainly retained at the membrane fraction of colorectal cancer cells (FIG. 7).

The FIG. 7 illustrates the analysis of PMC79 cellular distribution after 48 h of incubation with IC₅₀ value by ICP-MS. Values represent mean±S.D. of at least three independent experiments.

### PMC79 Induces Alterations in Actin Cytoskeleton of Colorectal Cancer Cells

With the purpose of assessing the effects of PMC79 compound in the actin cytoskeleton structure of SW480 cells, the F-actin organization and morphology was analyzed using phalloidin. The cells were treated with IC₅₀ value of PMC79 for 48 h, and then were stained with Phalloidin-AlexaFluor® 568.

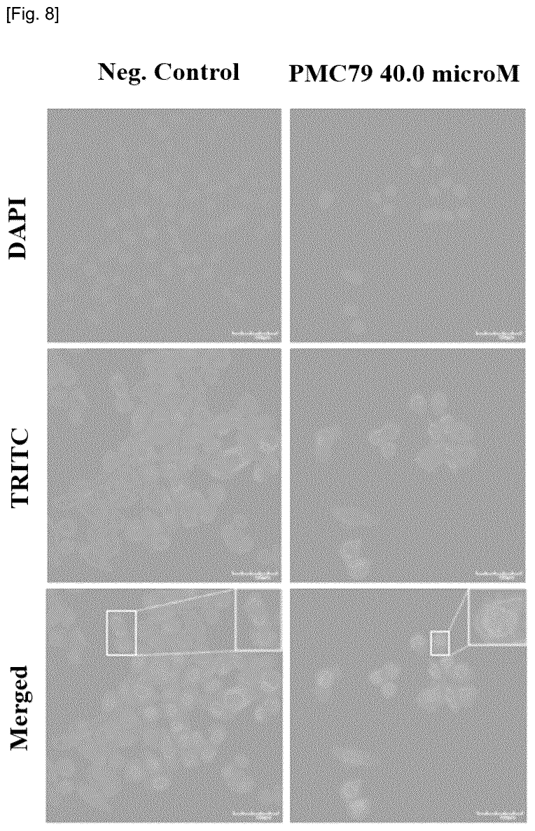

PMC79 appeared to affect cell-cell adhesion and intercellular contacts establishment, accompanied by alterations in cell phenotype, namely roundness and cell dispersion (FIG. 8). In addition, PMC79 also seemed to affect cytoskeleton organization inducing filopodia-like protrusions. It was also observed a decrease in the number of cells present in PMC79 condition in comparison with negative control. This might be due to the decrease in proliferation and increase of apoptosis induced by PMC79.

The FIG. 8 illustrates the analysis of F-actin staining performed using the IC₅₀ values of PMC79, for 48 h. Representative images (×600) of DAPI (4′,6-diamidino-2-phenylindole), Phalloidin-AlexaFluor 568 and merged were obtained in a fluorescence microscope. The results were obtained from at least three independent experiments.

PMC79 Inhibits the Expression of KRAS, AKT and ERK Proteins in Colorectal Cancer Cells with KRAS Mutation

KRAS downstream signaling pathways (MAPK-ERK and PI3K-AKT) are involved in the regulation of survival, proliferation and motility of CRC cells. Therefore, it is of utmost importance to understand whether PMC79 compound might affect KRAS signaling pathways by analyzing the expression of KRAS and downstream molecules AKT and ERK proteins, involved in PI3K and MAPK pathways, respectively.

As a comparison cisplatin and three other related ruthenium-derived compounds were used. Cisplatin is known to be a platinum drug widely used in cancer therapy. PMC78, LCR134 and LCR220 are ruthenium-cyclopentadienyl compounds distinct from PMC79 and with different functionalization to target cancer cells.

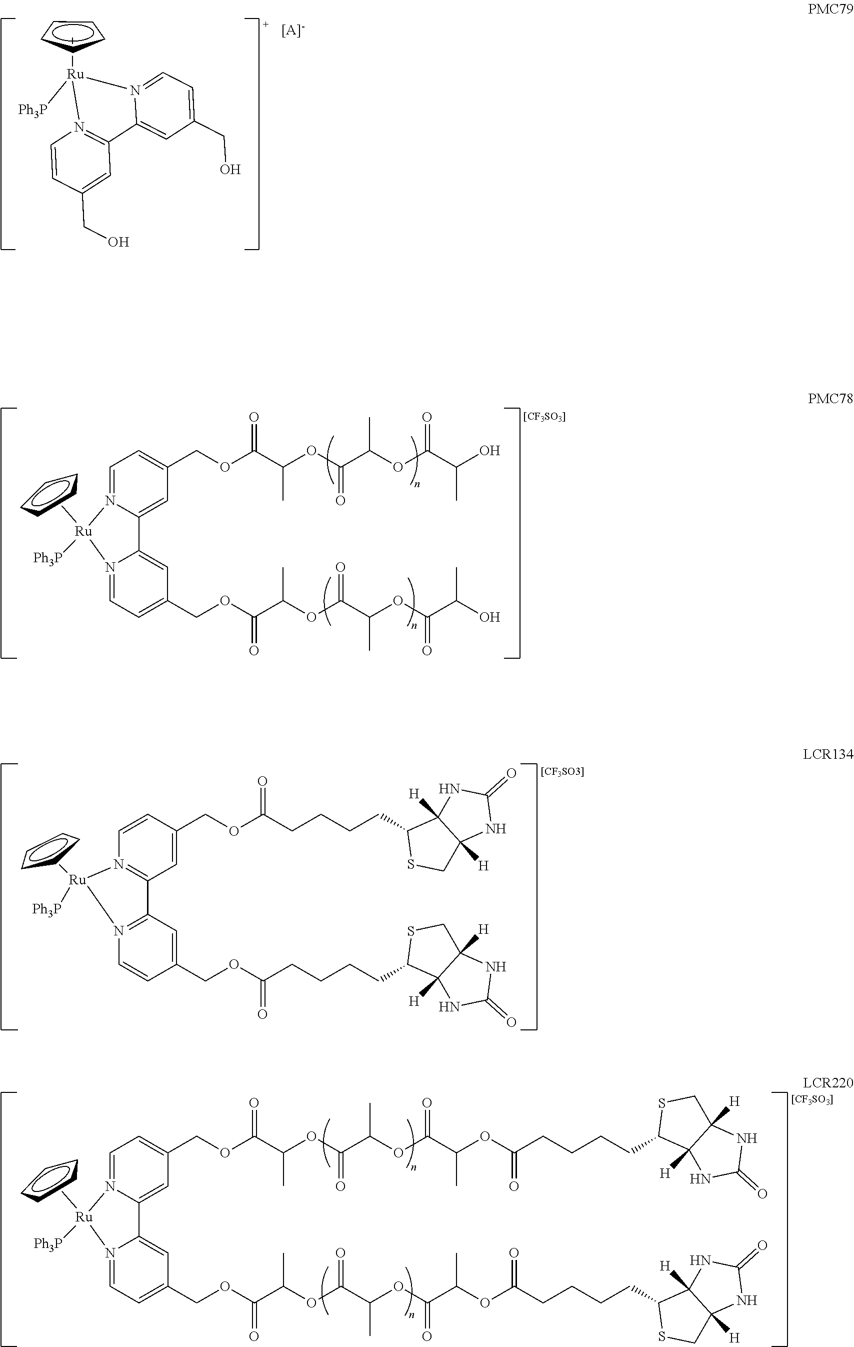

SW480 cells were exposed for 48 h, at the IC₅₀ concentration of each compound before protein extraction and Western blot analysis.

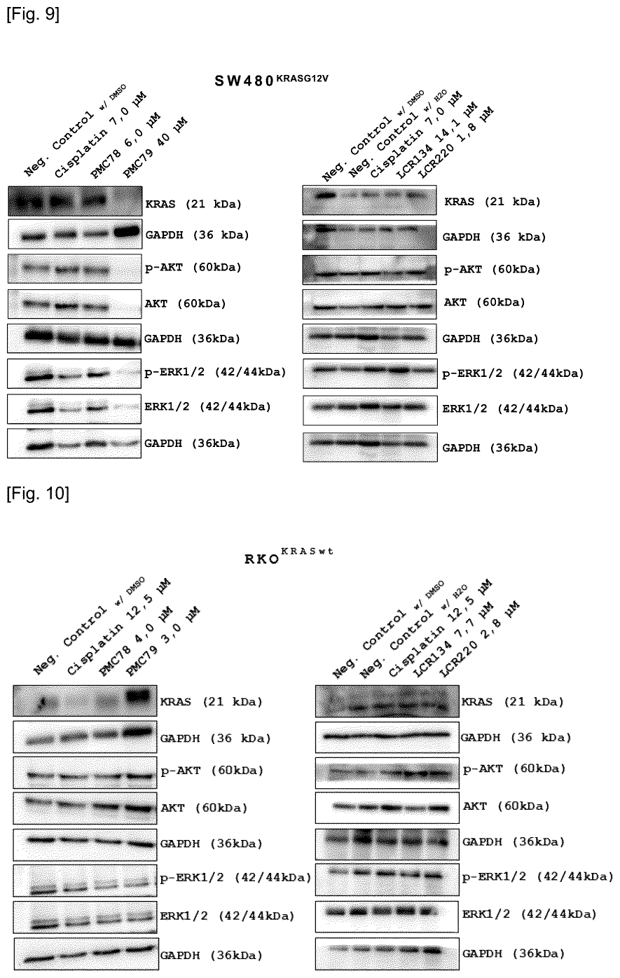

The results demonstrated that PMC79 was able to inhibit to a very high extent the expression of KRAS, PERK, total-ERK, pAKT and total-AKT in SW480 cells harboring KRAS mutation (FIG. 9). This was not observed when cells were exposed to cisplatin or the other ruthenium-derived compounds (PMC78, LCR134, LCR220) revealing that PMC79 is able to inhibit mutated KRAS and blocking MAPK-ERK and PI3K-AKT, the most important signaling pathways in CRC carcinogenesis.

The FIG. 9 illustrates a Western blot analysis of KRAS, pAKT, total-AKT, pERK and total-ERK proteins, in SW480 cells, after 48 h of incubation with IC₅₀ values of Cisplatin, PMC78, PMC79, LCR134 and LCR220. GAPDH immunoblot was used as a loading control. The results were obtained from at least three independent experiments.

The effect of PMC79 in RKO cell line harboring a wild-type KRAS was also studied and no effect on the expression of KRAS, PERK, total-ERK, pAKT and total-AKT proteins was observed. The same was also observed for cisplatin and PMC78, LCR134, LCR220 compounds (FIG. 10).

The FIG. 10 illustrates a Western blot analysis of KRAS, pAKT, total-AKT, pERK and total-ERK proteins in RKO cells, after 48 h of incubation with IC₅₀ values of Cisplatin, PMC78, PMC79, LCR134 and LCR220. GAPDH immunoblot was used as a loading control. The results were obtained from at least three independent experiments.

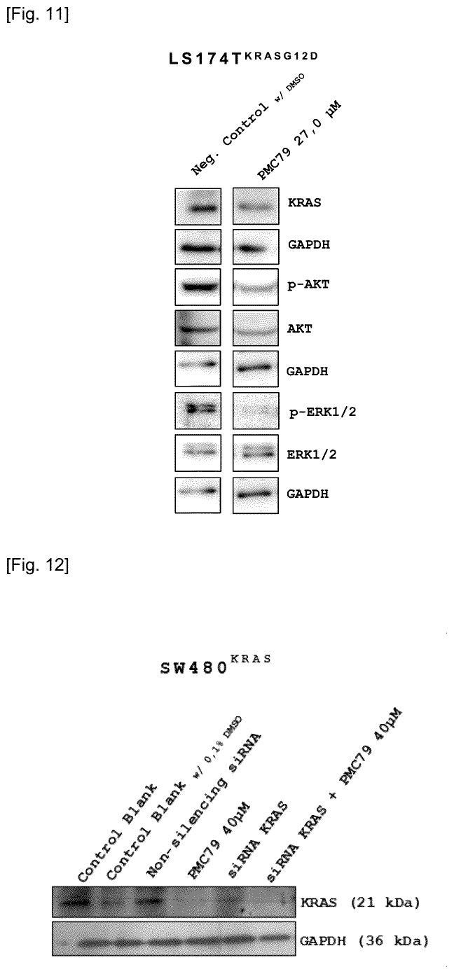

The effect of PMC79 in a different cell line with KRAS mutation was also evaluated using LS174T cells. The results demonstrated that PMC79 was also able to inhibit the expression of KRAS, PERK, total-ERK, pAKT and total-AKT in this cell line (FIG. 11).

The FIG. 11 illustrates a Western blot analysis of KRAS, pAKT, total-AKT, pERK and total-ERK proteins, in LS174T cells, after 48 h of incubation with PMC79. GAPDH immunoblot was used as a loading control. The results were obtained from at least three independent experiments.

To compare the KRAS inhibition by PMC79 with a molecule known to specifically target and inhibit KRAS we used a siRNA for KRAS in order to compare the degree of inhibition induced by both molecules (Alves et al., 2015). The results demonstrated that PMC79 showed an inhibition in the expression levels of KRAS similar to the specific siRNA for KRAS (FIG. 12).

The FIG. 12 illustrates a Western blot analysis of KRAS protein in SW480 cells. SW480 cells were left non-transfected (control blank) or transfected with either control siRNA or siRNA targeted against KRAS. After 6 h of transfection, cells were incubated with IC₅₀ value of PMC79 for 48 h. GAPDH immunoblot was used as a loading control. The results were obtained from at least three independent experiments.

Overall, these results demonstrated that PMC79 is a strong inhibitor of mutated KRAS, specially KRAS^{G12V} and KRAS^{G12D} mutations.

### PMC79 Decreases the Levels of all Proteins of KRAS Signaling Pathways

Taking into consideration the results obtained on the inhibition of expression of some proteins involved in KRAS signaling pathways, a proteomic analysis was performed using SW480 extracts treated with IC₅₀ values of PMC79 for 48 h.

In accordance with the previous results, the proteomic study revealed that all proteins involved in KRAS signaling pathways are less abundant in PMC79 than in the control condition, namely EGFR, KRAS, MEK 1, MEK 2, ERK1, PIK3, AKT1, AKT2 and MTOR (Table 2). Only two proteins showed a significant decrease, namely MEK1 (**P≤0,01), AKT1 (*P≤0,05).

Table 2 shows the proteomic analysis of proteins involved in MAPK and PI3K signaling pathways, in SW480 cells, after 48 h of incubation with IC₅₀ of PMC79. Values represent mean of at least three independent experiments.

TABLE 2

| | Abundance Ratio (log2): | Abundance Ratio P-Value: |
|---|---|---|
| Protein | (pmc79)/(Control) | (pmc79)/(Control) |
| MEK1 | −0.542145667 | 0.0025 |
| AKT1 | −0.403896333 | 0.0212 |
| EGFR | −0.948012667 | 0.2443 |
| KRAS | −0.401868333 | 0.2602 |
| MTOR | −1.326296667 | 0.2904 |
| AKT2 | −0.250630333 | 0.3101 |
| MEK2 | −0.236476 | 0.3125 |
| ERK1 | −0.360106 | 0.3573 |
| PIK3R4 | −0.555468333 | 0.6147 |
| PIK3C3 | −0.17947 | |
| PIK3R2 | −0.950935 | |

The proteomic study supported the previous results confirming that PMC79 inhibits the expression levels of KRAS downstream signaling proteins.

### Discussion

In the present invention, we demonstrated that an organometallic ruthenium compound revealed promising anticancer properties for therapy of CRC with KRAS^{G12V} and KRAS^{G12D} mutations. PMC79 revealed to be selective in inhibiting the survival of CRC cells, showing a selectivity index of 1.1. This value indicates that the anti-cancer effect on cancer cells surpassed normal cells that will not be damaged by PMC79 (Ferreira et al., 2017).

The anticancer effect of a compound is largely based on the ability of the drug to inhibit proliferation, metastasis and induce apoptotic cell death in cancer cells. These abilities are mostly correlated with good results in most preclinical and clinical studies (Kim, 2005).

Overall, the results showed that PMC79 inhibit proliferation in CRC with KRAS mutation by decreasing colony formation ability, inducing a G0/G1 cell cycle arrest and increasing CFSE fluorescence. Moreover, PMC79 induce an apoptotic cell death as demonstrated by increasing in Annexin V and TUNEL positive cells with the presence of apoptotic bodies.

In addition, PMC79 induces alterations in the actin cytoskeleton of CRC cells. The cytoskeleton is known to be responsible for several cellular processes such as cell division, cellular motility, and cell death (Fletcher & Mullins, 2010). PMC79 revealed to affect cell cytoskeleton organization with cell dispersion and evident filopodia-like protrusions (Bornschl, 2013). These motility like-structures are related, among other things, with cellular retraction. The results of wound healing assay also indicated a decrease of motility in cells exposed to PMC79 which support the role of filopodia-like protrusions in the inhibition of cellular motility. Inhibition of motility is also important in order to inhibit the metastatic potential of CRC cells.

Here we showed that the preferred compound according to the invention (PMC79) presents anticancer properties, which are accomplished by decreasing proliferation rate and cell migration while increasing apoptosis, three important hallmarks of cancer (Hanahan, 2022). These phenotypic properties associated to PMC79 are determinant on its effect as a potent therapeutic agent against CRC with KRAS mutation, what will decrease the oncogenic potential of these CRC and thus inhibit tumor progression.

KRAS downstream signaling pathways (MAPK-ERK and PI3K-AKT) are involved in survival and proliferative processes playing an important role in CRC carcinogenesis and influencing the progression of this disease. The major goal of any therapy is to inactivate relevant proteins involved in signaling pathways which might simultaneously result in inhibition of cellular proliferation, metastasis and induction of apoptosis.

Nothing was known about the interaction of ruthenium-derived compounds with KRAS signaling pathways (Mahmud et al., 2021). Here we studied the expression levels of KRAS and downstream signaling proteins and showed for the first time that an organometallic ruthenium compound according to the invention, namely PMC79, inhibited the expression of KRAS signaling.

The intracellular distribution of PMC79 showed that this compound is preferentially distributed in the membrane fraction what could explain the KRAS inhibitory effect, since KRAS localizes at the membrane level and needs to be attached to the cell membrane to be activated (Cazzanelli et al., 2018; László et al., 2021).

KRAS-activating mutations are the most frequent oncogenic alterations in CRC, but the therapeutic options in these types of cancers are scarce (Laurent-Puig et al., 2009; Pereira et al., 2022). A series of strategies tried to indirectly target KRAS, although these efforts have not been successful in clinical trials over the past 30 years (Ryan & Corcoran, 2018). Recently, two direct KRAS^{G12C} inhibitors, namely sotorasib and adagrasib were developed, which act by selectively forming a covalent bond with cysteine 12 within the switch-II pocket of KRAS^{G12C} protein, thereby locking KRAS in the inactive state (Briere et al., 2021; Canon et al., 2019; Fell et al., 2020; Hallin et al., 2020; Lanman et al., 2020; Skoulidis et al., 2021; Liu et al., 2021). Preclinical studies have shown that sotorasib and adagrasib selectively impair the viability of KRAS^{G12C} mutant cell lines of NSCLC and PDAC, decrease proliferation, induce apoptosis and also inhibit the levels of p-ERK protein (Canon et al., 2019; Hallin et al., 2020). However, neither sotorasib nor adagrasib affect PI3K signaling, which provides an explanation for the acquisition of resistance to these KRAS^{G12C} inhibitors (Hallin et al., 2020). Moreover, sotorasib and adagrasib do not affect cell lines with other KRAS mutations in vitro and in vivo (Briere et al., 2021; Canon et al., 2019; Fell et al., 2020; Hallin et al., 2020; Lanman et al., 2020; Skoulidis et al., 2021; Liu et al., 2021).

In addition to sotorasib and adagrasib, the development of other covalent inhibitors of KRAS^{G12C}, such as ARS-1620, GDC-6036, D-1553, 1_AM, and ARS-853 may also provide new opportunities for selective targeting of various advanced solid tumors carrying KRAS^{G12C} mutations (Janes et al., 2018; Molina-Arcas et al., 2019; Ostrem et al., 2013; Patricelli et al., 2016; Liu et al., 2021; Lito et al., 2016; Zeng et al., 2017). Despite the discovery of all these inhibitors, KRAS^{G12C} mutation is not a hotspot mutation in CRC, being present in only 3% of CRCs with KRAS mutation, which explains why they are only being used in the clinics for treatment of NSCLC (László et al., 2021; Liu et al., 2021).

More recently, a KRAS^{G12D} inhibitor has been discovered however its application has only been studied in vitro and in vivo for the PDAC model (Xiaolun Wang et al., 2022; Kemp S B, 2023).

In the present invention, it was demonstrated that PMC79 inhibits KRAS and both KRAS signaling pathways (MAPK and PI3K) in cells harboring KRAS mutation but not in cells with KRAS wild-type (FIG. 10). This simultaneous inhibition of MAPK and PI3K pathways is an advantage over the known inhibitors and may prevent potential resistance problems in the future.

In addition, it was also demonstrated that PMC79 inhibits KRAS in a similar way than a siRNA designed to specifically target KRAS.

The inhibition of KRAS using siRNA has been studied as an alternative approach to target KRAS mutations in CRC, although the difficulty in finding efficient delivery systems that increase cellular uptake and reduce off-target effects of RNAi has been a challenge (Jebelli et al., 2021).

Overall, the results gathered here support the use of PMC79 as a new specific and potent KRAS signaling inhibitor agent in CRC harboring KRAS mutations, specially KRAS^{G12V} and KRAS^{G12D} mutations.

As mentioned above, KRAS mutations are particularly common in CRC (40%) but are also frequent in PDAC (80%), NSCLC (35%) and ovarian and endometrial cancer (10%) (Christensen et al., 2020; Haigis, 2017; Timar & Kashofer, 2020). Given the difficulties in finding therapies that specifically target KRAS, sotorasib and adagrasib have brought new solutions for the treatment of cancers with KRAS mutation by inhibiting KRAS^{G12C} in NSCLC and PDAC (Briere et al., 2021; Canon et al., 2019; Fell et al., 2020; Hallin et al., 2020; Lanman et al., 2020; Skoulidis et al., 2021; Liu et al., 2021). Considering that these inhibitors have obtained good results in cell lines from different cancer models, our results support the hypothesis that PMC79 may also inhibit KRAS in other types of cancers with KRAS mutation, such as PDAC, NSCLC and ovarian and endometrial cancer.

As used in this description, the expressions “around” and “approximately” refer to an interval of values of more or less 10% of the number specified.

As used in this description, the expression “substantially” means that the real value is within the interval of around 10% of the desired value, variable or related limit, particularly within around 5% of the desired value, variable or related limit or specially within the 1% of the desired value, variable or related limit.

The subject matter described above is provided as an illustration of the present invention and, therefore, cannot be interpreted so as to limit it. The terminology used herein with the purpose of describing preferred embodiments of the present invention, must not be interpreted to limit the invention. As used in the specification, the definite and indefinite articles, in their singular form, aim at the interpretation of also including the plural forms, unless the context of the description indicates, explicitly, the contrary. It will be understood that the expressions “comprise” and “include”, when used in this description, specify the presence of the characteristics, the elements, the components, the steps and the related operations, however, they do not exclude the possibility of other characteristics, elements, components, steps and operations also being contemplated.

All the alterations, providing that they do not modify the essential characteristics of the claims that follow, must be considered as being within the scope of protection of the present invention.

### CITATION LIST

Follows the list of citations:

### PATENT LITERATURE

PTL1: WO2016087932A2 (Anselmo Viegas Garcia et al), published on 2016 Jun. 9.

### Non-Patent Literature

NPL1: Alsaab, H. O., Alghamdi, M. S., Alotaibi, A. S., Alzhrani, R., Alwuthaynani, F., Althobaiti, Y. S., Almalki, A. H., Sau, S., & lyer, A. K. (2020). Progress in clinical trials of photodynamic therapy for solid tumors and the role of nanomedicine. Cancers, 12(10), 1-26. https://doi.org/10.3390/cancers12102793
NPL2: Alves, S., Castro, L., Fernandes, M. S., Francisco, R., Castro, P., Priault, M., Chaves, S. R., Moyer, M. P., Oliveira, C., Seruca, R., Corte-Real, M., Sousa, M. J., & Preto, A. (2015). Colorectal cancer-related mutant KRAS alleles function as positive regulators of autophagy. Oncotarget, 6(31), 30787-30802. https://doi.org/10.18632/oncotarget.5021
NPL3: Beer L A, Liu P, Ky B, Barnhart K T, Speicher D W. Efficient Quantitative Comparisons of Plasma Proteomes Using Label-Free Analysis with MaxQuant. Methods Mol Biol. 2017; 1619:339-352. doi: 10.1007/978-1-4939-7057-5_23. PMID: 28674895; PMCID: PMC5575765.
NPL4: Berg, M., & Soreide, K. (2012). EGFR and downstream genetic alterations in KRAS/BRAF and PI3K/AKT pathways in colorectal cancer: implications for targeted therapy. Discovery Medicine, 14(76), 207-214. http://www.ncbi.nlm.nih.gov/pubmed/23021375
NPL5: Bergamo, A., Gaiddon, C., Schellens, J. H. M., Beijnen, J. H., & Sava, G. (2012). Approaching tumour therapy beyond platinum drugs: Status of the art and perspectives of ruthenium drug candidates. In Journal of Inorganic Biochemistry (Vol. 106, Issue 1, pp. 90-99). https://doi.org/10.1016/j.jinorgbio.2011.09.030
NPL6: Bornschl, T. (2013). How Filopodia Pull: What We Know About the Mechanics and Dynamics of Filopodia. Cytoskeleton. https://doi.org/10.1002/cm.21130
NPL7: Briere, D. M., Li, S., Calinisan, A., Sudhakar, N., Aranda, R., Hargis, L., Peng, D. H., Deng, J., Engstrom, L. D., Hallin, J., Gatto, S., Fernandez-Banet, J., Pavlicek, A., Wong, K. K., Christensen, J. G., & Olson, P. (2021). The KRAS^{G12C} inhibitor MRTX849 reconditions the tumor immune microenvironment and sensitizes tumors to checkpoint inhibitor therapy. Molecular Cancer Therapeutics, 20(6), 975-985. https://doi.org/10.1158/1535-7163.MCT-20-0462
NPL8: Canon, J., Rex, K., Saiki, A. Y., Mohr, C., Cooke, K., Bagal, D., Gaida, K., Holt, T., Knutson, C. G., Koppada, N., Lanman, B. A., Werner, J., Rapaport, A. S., San Miguel, T., Ortiz, R., Osgood, T., Sun, J. R., Zhu, X., McCarter, J. D., Lipford, J. R. (2019). The clinical KRAS (G12C) inhibitor AMG 510 drives anti-tumour immunity. Nature, 575(7781), 217-223. https://doi.org/10.1038/s41586-019-1694-1
NPL9: Cazzanelli, G., Moreira, T., Ferro, S., Azevedo-Silva, J., Nogueira, E., & Preto, A. (2016). Colorectal Cancer Therapeutic Approaches: From Classical Drugs to New Nanoparticles. In Frontiers in Anti-Cancer Drug Discovery. https://doi.org/10.2174/9781681082516116070004
NPL10: Cazzanelli, G., Pereira, F., Alves, S., Francisco, R., Azevedo, L., Carvalho, P. D., Almeida, A., Côrte-Real, M., Oliveira, M. J., Lucas, C., Sousa, M. J., & Preto, A. (2018). The yeast Saccharomyces cerevisiae as a model for understanding RAS proteins and their role in human tumorigenesis. In Cells (Vol. 7, Issue 2). https://doi.org/10.3390/cells7020014
NPL11: Chiva C. et al., “QCloud: A cloud-based quality control system for mass spectrometry-based proteomics laboratories,” PLOS One, vol. 13, no. 1, January 2018, doi: 10.1371/journal.pone.0189209.
NPL12: Christensen, J. G., Olson, P., Briere, T., Wiel, C., & Bergo, M. O. (2020). Targeting KRAS^{G12C}-mutant cancer with a mutation-specific inhibitor. In Journal of Internal Medicine (Vol. 288, Issue 2, pp. 183-191). Blackwell Publishing Ltd. https://doi.org/10.1111/joim.13057
NPL13: Côrte-Real, L., Karas, B., Brás, A. R., Pilon, A., Avecilla, Fernando Marques, F., Preto, A., Buckley, B., Cooper, K., Doherty, C., Garcia, M., & Valente, A. (2019). Ruthenium-cyclopentadienyl bipyridine-biotin based compounds: synthesis and biological effect. Inorganic Chemistry, 59, 9135-9149. https://doi.org/10.1021/acs.inorgchem.9b00735
NPL14: Côrte-Real, L., Karas, B., Gírio, P., Moreno, A., Avecilla, F., Marques, F., Buckley, B. T., Cooper, K. R., Doherty, C., Falson, P., Garcia, M. H., & Valente, A. (2019). Unprecedented inhibition of P-gp activity by a novel ruthenium-cyclopentadienyl compound bearing a bipyridine-biotin ligand. European Journal of Medicinal Chemistry, 163, 853-863. https://doi.org/https://doi.org/10.1016/j.ejmech.2018.12.022
NPL15: Côrte-Real, L., Matos, A. P., Alho, I., Morais, T. S., Tomaz, A. I., Garcia, M. H., Santos, I., Bicho, M. P., & Marques, F. (2013). Cellular uptake mechanisms of an antitumor ruthenium compound: The endosomal/lysosomal system as a target for anticancer metal-based drugs. Microscopy and Microanalysis, 19(5), 1122-1130. https://doi.org/10.1017/S143192761300175X
NPL16: Fell, J. B., Fischer, J. P., Baer, B. R., Blake, J. F., Bouhana, K., Briere, D. M., Brown, K. D., Burgess, L. E., Burns, A. C., Burkard, M. R., Chiang, H., Chicarelli, M. J., Cook, A. W., Gaudino, J. J., Hallin, J., Hanson, L., Hartley, D. P., Hicken, E. J., Hingorani, G. P., . . . Marx, M. A. (2020). Identification of the Clinical Development Candidate MRTX849, a Covalent KRAS^{G12C} Inhibitor for the Treatment of Cancer. Journal of Medicinal Chemistry, 63(13), 6679-6693. https://doi.org/10.1021/acs.jmedchem.9b02052
NPL17: Ferreira, M., Assunção, L. S., Silva, A. H., Filippin-Monteiro, F. B., Creczynski-Pasa, T. B., & Sá, M. M. (2017). Allylic isothiouronium salts: The discovery of a novel class of thiourea analogues with antitumor activity. European Journal of Medicinal Chemistry, 129, 151-158. https://doi.org/10.1016/j.ejmech.2017.02.013
NPL18: Fletcher, D. A., & Mullins, R. D. (2010). Cell mechanics and the cytoskeleton. Nature, 463(7280), 485-492. https://doi.org/10.1038/nature08908.Cell
NPL19: Haigis, K. M. (2017). KRAS Alleles: The Devil Is in the Detail. Trends in Cancer, 3(10), 686-697. https://doi.org/10.1016/J.TRECAN.2017.08.006
NPL20: Hallin, J., Engstrom, L. D., Hargi, L., Calinisan, A., Aranda, R., Briere, D. M., Sudhakar, N., Bowcut, V., Baer, B. R., Ballard, J. A., Burkard, M. R., Fell, J. B., Fischer, J. P., Vigers, G. P., Xue, Y., Gatto, S., Fernandez-Banet, J., Pavlicek, A., Velastagui, K., . . . Christensen, J. G. (2020). The KRAS^{G12C} inhibitor MRTX849 provides insight toward therapeutic susceptibility of KRAS-mutant cancers in mouse models and patients. Cancer Discovery, 10(1), 54-71. https://doi.org/10.1158/2159-8290.CD-19-1167
NPL21: Hanahan, D. (2022). Hallmarks of Cancer: New Dimensions. Cancer Discovery, 12(1), 31-46. https://doi.org/10.1158/2159-8290.CD-21-1059
NPL22: Hong, S., Kim, S., Kim, H. Y., Kang, M., Jang, H. H., & Lee, W. S. (2016). Targeting the PI3K signaling pathway in KRAS mutant colon cancer. Cancer Medicine, 5(2), 248-255. https://doi.org/10.1002/cam4.591
NPL23: Janes, M. R., Zhang, J., Li, L. S., Hansen, R., Peters, U., Guo, X., Chen, Y., Babbar, A., Firdaus, S. J., Darjania, L., Feng, J., Chen, J. H., Li, S., Li, S., Long, Y. O., Thach, C., Liu, Y., Zarieh, A., Ely, T., . . . Liu, Y. (2018). Targeting KRAS Mutant Cancers with a Covalent G12C-Specific Inhibitor. Cell, 172(3), 578-589.e17. https://doi.org/10.1016/j.cell.2018.01.006
NPL24: Jebelli, A., Baradaran, B., Mosafer, J., Baghbanzadeh, A., Mokhtarzadeh, A., & Tayebi, L. (2021). Recent developments in targeting genes and pathways by RNAi-based approaches in colorectal cancer. Medicinal Research Reviews, 41(1), 395-434. https://doi.org/10.1002/med.21735
NPL25: Kemp S B, Cheng N, Markosyan N, Sor R, Kim I K, Hallin J, Shoush J, Quinones L, Brown N V, Bassett J B, Joshi N, Yuan S, Smith M, Vostrejs W P, Perez-Vale K Z, Kahn B, Mo F, Donahue T R, Radu C G, Clendenin C, Christensen J G, Vonderheide R H, Stanger B Z. Efficacy of a Small-Molecule Inhibitor of KRAS^{G12D} in Immunocompetent Models of Pancreatic Cancer. Cancer Discov. 2023 Feb. 6; 13(2): 298-311. doi: 10.1158/2159-8290.CD-22-1066. PMID: 36472553; PMCID: PMC9900321.
NPL26: Kim, R. (2005). Recent advances in understanding the cell death pathways activated by anticancer therapy. Cancer, 103(8), 1551-1560. https://doi.org/10.1002/cncr.20947
NPL27: Lanman, B. A., Allen, J. R., Allen, J. G., Amegadzie, A. K., Ashton, K. S., Booker, S. K., Chen, J. J., Chen, N., Frohn, M. J., Goodman, G., Kopecky, D. J., Liu, L., Lopez, P., Low, J. D., Ma, V., Minatti, A. E., Nguyen, T. T., Nishimura, N., Pickrell, A. J., . . . Cee, V. J. (2020). Discovery of a Covalent Inhibitor of KRAS^{G12C} (AMG 510) for the Treatment of Solid Tumors. Journal of Medicinal Chemistry, 63(1), 52-65. https://doi.org/10.1021/acs.jmedchem.9b01180
NPL28: László, L., Kurilla, A., Takács, T., Kudlik, G., Koprivanacz, K., Buday, L., & Vas, V. (2021). Recent updates on the significance of kras mutations in colorectal cancer biology. Cells, 10(3), 1-19. https://doi.org/10.3390/cells10030667
NPL29: Laurent-Puig, P., Cayre, A., Manceau, G., Buc, E., Bachet, J.-B., Lecomte, T., Rougier, P., Lievre, A., Landi, B., Boige, V., Ducreux, M., Ychou, M., Bibeau, F., Bouché, O., Reid, J., Stone, S., & Penault-Llorca, F. (2009). Analysis of PTEN, BRAF, and EGFR Status in Determining Benefit From Cetuximab Therapy in Wild-Type KRAS Metastatic Colon Cancer. Journal of Clinical Oncology, 27(35), 5924-5930. https://doi.org/10.1200/JCO.2008.21.6796
NPL30: Lee, J. J., & Sun, W. (2016). Options for Second-Line Treatment in Metastatic Colorectal Cancer. 14(1), 46-54.
NPL31: Li, Z.-N., Zhao, L., Yu, L.-F., & Wei, M.-J. (2020). BRAF and KRAS mutations in metastatic colorectal cancer: future perspectives for personalized therapy. Gastroenterology Report, 8(3), 192-205. https://doi.org/10.1093/gastro/goaa022
NPL32: Lito, P., Solomon, M., Li, L. S., Hansen, R., & Rosen, N. (2016). Allele-specific inhibitors inactivate mutant KRAS G12C by a trapping mechanism. Science, 351(6273), 604-608. https://doi.org/10.1126/science.aad6204
NPL33: Liu, J., Kang, R., & Tang, D. (2021). The KRAS-G12C inhibitor: activity and resistance. Cancer Gene Therapy, 2021 (July). https://doi.org/10.1038/s41417-021-00383-9
NPL34: Mahmud, K. M., Niloy, M. S., & Shakil, S. (2021). Ruthenium Complexes: An Alternative to Platinum Drugs in Colorectal Cancer Treatment. 1-30.
NPL35: Molina-Arcas, M., Moore, C., Rana, S., van Maldegem, F., Mugarza, E., Romero-Clavijo, P., Herbert, E., Horswell, S., Li, L. S., Janes, M. R., Hancock, D. C., & Downward, J. (2019). Development of combination therapies to maximize the impact of KRAS-G12C inhibitors in lung cancer. Science Translational Medicine, 11(510). https://doi.org/10.1126/scitranslmed.aaw7999
NPL36: Morais, T. S., Valente, A., Tomaz, A. I., Marques, F., & Garcia, M. H. (2016). Tracking antitumor metallodrugs: promising agents with the Ru(II)- and Fe(II)-cyclopentadienyl scaffolds. Future Madicinal Chemistry, 8(5), 527-544.
NPL37: Moreira, T., Francisco, R., Comsa, E., Duban-Deweer, S., Labas, V., Teixeira-Gomes, A. P., Combes-Soia, L., Marques, F., Matos, A., Favrelle, A., Rousseau, C., Zinck, P., Falson, P., Garcia, M. H., Preto, A., & Valente, A. (2019). Polymer “ruthenium-cyclopentadienyl” conjugates—New emerging anti-cancer drugs. European Journal of Medicinal Chemistry, 168, 373-384. https://doi.org/10.1016/j.ejmech.2019.02.061
NPL38: Moyer, M. P., Manzano, L. A., Merriman, R. L., & et al. (1996). NCM460, A NORMAL HUMAN COLON MUCOSAL EPITHELIAL CELL LINE. In Vitro Cell. Dev. Biol.-Animal, 32, 315-316. https://doi.org/https://doi.org/10.1007/BF02722955
NPL39: Nagasaka, M., Li, Y., Sukari, A., Ou, S. H. I., Al-Hallak, M. N., & Azmi, A. S. (2020). KRAS G12C Game of Thrones, which direct KRAS inhibitor will claim the iron throne? Cancer Treatment Reviews, 84 (December 2019), 101974. https://doi.org/10.1016/j.ctrv.2020.101974
NPL40: Olivella R. et al., “QCloud2: An Improved Cloud-based Quality-Control System for Mass-Spectrometry-based Proteomics Laboratories,” J Proteome Res, vol. 20, no. 4, pp. 2010-213 April 2021, doi: 10.1021/acs.jproteome.0c00853.
NPL41: Ostrem, J. M., Peters, U., Sos, M. L., Wells, J. A., & Shokat, K. M. (2013). K-Ras (G12C) inhibitors allosterically control GTP affinity and effector interactions. Nature, 503(7477), 548-551. https://doi.org/10.1038/nature12796
NPL42: Pardini, B., Kumar, R., Naccarati, A., Novotny, J., Prasad, R. B., Forsti, A., Hemminki, K., Vodicka, P., & Lorenzo Bermejo, J. (2011). 5-Fluorouracil-based chemotherapy for colorectal cancer and MTHFR/MTRR genotypes. British Journal of Clinical Pharmacology, 72(1), 162-163. https://doi.org/10.1111/j.1365-2125.2010.03892.x
NPL43: Patricelli, M. P., Janes, M. R., Li, L. S., Hansen, R., Peters, U., Kessler, L. v., Chen, Y., Kucharski, J. M., Feng, J., Ely, T., Chen, J. H., Firdaus, S. J., Babbar, A., Ren, P., & Liu, Y. (2016). Selective inhibition of oncogenic KRAS output with small molecules targeting the inactive state. Cancer Discovery, 6(3), 316-329. https://doi.org/10.1158/2159-8290.CD-15-1105
NPL44: Pereira, F., Ferreira, A., Reis, C. A., Sousa, M. J., Oliveira, M. J., & Preto, A. (2022). KRAS as a Modulator of the Inflammatory Tumor Microenvironment: Therapeutic Implications. Cells, 11(3), 398. https://doi.org/10.3390/cells11030398
NPL45: Y. Perez-Riverol et al., “The PRIDE database resources in 2022: A hub for mass spectrometry-based proteomics evidences,” Nucleic Acids Res, vol. 50, no. D1, pp. D543-D552, January 2022, doi: 10.1093/nar/gkab1038.
NPL46: Perkins D N, Pappin D J, Creasy D M, Cottrell J S. Probability-based protein identification by searching sequence databases using mass spectrometry data. Electrophoresis. 1999 December; 20(18): 3551-67. doi: 10.1002/(SICI) 1522-2683(19991201) 20:18<3551::AID-ELPS3551>3.0.CO;2-2. PMID: 10612281.
NPL47: Pilon, A., Brás, A. R., Côrte-Real, L., Avecilla, F., Costa, P. J., Preto, A., Helena Garcia, M., & Valente, A. (2020). A new family of iron (II)-cyclopentadienyl compounds shows strong activity against colorectal and triple negative breast cancer cells. Molecules, 25(7). https://doi.org/10.3390/molecules25071592
NPL48: Ryan, M. B., & Corcoran, R. B. (2018a). Therapeutic strategies to target RAS-mutant cancers. Nature Reviews Clinical Oncology, 15(11), 709-720. https://doi.org/10.1038/s41571-018-0105-0
NPL49: Ryan, M. B., & Corcoran, R. B. (2018b). Therapeutic strategies to target RAS-mutant cancers. Nature Reviews. Clinical Oncology, 15(11), 709-720. https://doi.org/10.1038/s41571-018-0105-0
NPL50: Skoulidis, F., Li, B. T., Dy, G. K., Price, T. J., Falchook, G. S., Wolf, J., Italiano, A., Schuler, M., Borghaei, H., Barlesi, F., Kato, T., Curioni-Fontecedro, A., Sacher, A., Spira, A., Ramalingam, S. S., Takahashi, T., Besse, B., Anderson, A., Ang, A., . . . Govindan, R. (2021). Sotorasib for Lung Cancers with KRAS p.G12C Mutation. New England Journal of Medicine, 384(25), 2371-2381. https://doi.org/10.1056/nejmoa2103695
NPL51: Sung, H., Ferlay, J., Siegel, R. L., Laversanne, M., Soerjomataram, I., Jemal, A., & Bray, F. (2021). Global Cancer Statistics 2020: GLOBOCAN Estimates of Incidence and Mortality Worldwide for 36 Cancers in 185 Countries. CA: A Cancer Journal for Clinicians, 71(3), 209-249. https://doi.org/10.3322/caac.21660
NPL52: Teixeira, R. G., Belisario, D. C., Fontrodona, X., Romero, I., Tomaz, A. I., Garcia, M. H., Riganti, C., & Valente, A. (2021). Unprecedented collateral sensitivity for cisplatin-resistant lung cancer cells presented by new ruthenium organometallic compounds. Inorganic Chemistry Frontiers, 8(8), 1983-1996. https://doi.org/10.1039/d0qi01344g
NPL53: Teixeira, R. G., Brás, A. R., Côrte-Real, L., Tatikonda, R., Sanches, A., Robalo, M. P., Avecilla, F., Moreira, T., Garcia, M. H., Haukkac, M., Preto, A., & Valente, A. (2018). Novel ruthenium methylcyclopentadienyl complex bearing a bipyridine perfluorinated ligand showing strong activity towards colorectal cancer cell lines. European Journal of Medicinal Chemistry, 503-514. https://doi.org/10.1016/j.ejmech.2017.11.059
NPL54: Temraz, S., Mukherji, D., & Shamseddine, A. (2015). Dual inhibition of MEK and PI3K pathway in KRAS and BRAF mutated colorectal cancers. International Journal of Molecular Sciences, 16(9), 22976-22988. https://doi.org/10.3390/ijms160922976
NPL55: Thota, S., Rodrigues, D. A., Crans, D. C., & Barreiro, E. J. (2018). Ru(II) Compounds: Next-Generation Anticancer Metallotherapeutics? [Review-article]. Journal of Medicinal Chemistry, 61(14), 5805-5821. https://doi.org/10.1021/acs.jmedchem.7b01689
NPL56: Timar, J., & Kashofer, K. (2020). Molecular epidemiology and diagnostics of KRAS mutations in human cancer. In Cancer and Metastasis Reviews (Vol. 39, Issue 4, pp. 1029-1038). Springer. https://doi.org/10.1007/s10555-020-09915-5
NPL57: Valente, A., Morais, T. S., Teixeira, R. G., Matos, C. P., Tomaz, A. I., & Garcia, M. H. (2021). Chapter 6-Ruthenium and iron metallodrugs: new inorganic and organometallic complexes as prospective anticancer agents. In E. J. M. Hamilton (Ed.), Synthetic Inorganic Chemistry (pp. 223-276). Elsevier. https://doi.org/https://doi.org/10.1016/B978-0-12-818429-5.00010-7
NPL58: Van Cutsem, E., Cervantes, A., Adam, R., Sobrero, A., Van Krieken, J. H., Aderka, D., Aranda Aguilar, E., Bardelli, A., Benson, A., Bodoky, G., Ciardiello, F., D'Hoore, A., Diaz-Rubio, E., Douillard, J. Y., Ducreux, M., Falcone, A., Grothey, A., Gruenberger, T., Haustermans, K., . . . Arnold, D. (2016). ESMO consensus guidelines for the management of patients with metastatic colorectal cancer. Annals of Oncology, 27(8), 1386-1422. https://doi.org/10.1093/annonc/mdw235
NPL59: Velma, V., Dasari, S. R., & Tchounwou, P. B. (2016). Low doses of cisplatin induce gene alterations, cell cycle arrest, and apoptosis in human promyelocytic leukemia cells. Biomarker Insights, 11, 113-121. https://doi.org/10.4137/Bmi.s39445
NPL60: Xiaolun Wang, Shelley Allen, James F. Blake, Vickie Bowcut, David M. Briere, Andrew Calinisan, Joshua R. Dahlke, Jay B. Fell, John P. Fischer, Robin J. Gunn, Jill Hallin, Jade Laguer, J. David Lawson, James Medwid, Brad Newhouse, Phong Nguyen, Jacob M. O'Leary, Peter Olson, Spencer Pajk, Lisa Rahbaek, Mareli Rodriguez, Christopher R. Smith, Tony P. Tang, Nicole C. Thomas, Darin Vanderpool, Guy P. Vigers, James G. Christensen, and Matthew A. Marx (2022). Identification of MRTX1133, a Noncovalent, Potent, and Selective KRAS^{G12D} Inhibitor. Journal of Medicinal Chemistry, 65(4), 3123-3133. doi: 10.1021/acs.jmedchem.1c01688
NPL61: Zaniboni, A. (2015). New active drugs for the treatment of advanced colorectal cancer. World Journal of Gastrointestinal Surgery, 7(12), 356-359. https://doi.org/10.4240/wjgs.v7.i12.356
NPL62: Zeng, M., Lu, J., Li, L., Feru, F., Quan, C., Gero, T. W., Ficarro, S. B., Xiong, Y., Ambrogio, C., Paranal, R. M., Catalano, M., Shao, J., Wong, K. K., Marto, J. A., Fischer, E. S., Janne, P. A., Scott, D. A., Westover, K. D., & Gray, N. S. (2017). Potent and Selective Covalent Quinazoline Inhibitors of KRAS G12C. Cell Chemical Biology, 24(8), 1005-1016.e3. https://doi.org/10.1016/j.chembiol.2017.06.017

1. An organometallic ruthenium compound characterized by having the general formula (I): 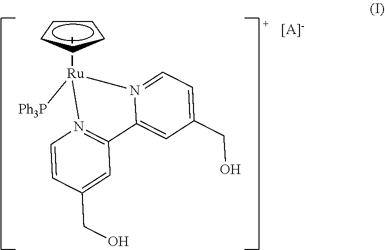
wherein:
A is selected from the group consisting of CF₃SO₃, PF₆, Cl, BPh₄, NO₃ or BF₄;
said organometallic ruthenium compound of formula (I) being in all the possible racemic, and stereoisomeric forms;
for use in the prevention and/or treatment of diseases and/or conditions related to colorectal cancer, pancreatic ductal adenocarcinoma, non-small cell lung cancer, and ovarian and endometrial cancer in an individual.

2. The organometallic ruthenium compound for use, according to claim 1, wherein the organometallic ruthenium compound of formula (I) is:
({2,2′-bipyridine}-4,4′-diyldimethanol-k²N, N′)(η⁵-cyclopentadienyl) (triphenylphosphane) ruthenium (II) triflate.

3. The organometallic ruthenium compound for use, according to claim 1, wherein the individual is a mammal, in particular a human.

4. The organometallic ruthenium compound for use, according to claim 1, wherein it is in a composition comprising a pharmaceutically acceptable excipient.

5. The organometallic ruthenium compound for use, according to claim 4, wherein the composition is administered by oral, rectal or parenteral injection route, preferably by parenteral route.

6. The organometallic ruthenium compound for use, according to claim 5, wherein said parenteral administration is selected from the group consisting of intravenous administration, intramuscular administration, intradermal administration, or subcutaneous administration.

7. The organometallic ruthenium compound for use, according to claim 4, wherein the composition is in a form selected from the group consisting of tablets, pills, powders, lozenges, sachets, cachets, elixirs, suspensions, emulsions, solutions, syrups, aerosols, sprays, gels, soft and hard gelatin capsules, suppositories, sterile injectable solutions, or sterile packages powders.

8. The organometallic ruthenium compound for use, according to claim 1, by inhibiting KRAS and downstream signaling pathways MAPK and PI3K.

9. The organometallic ruthenium compound, for use according to claim 8, wherein said KRAS and downstream signaling pathways MAPK and PI3K results in inhibition of cellular proliferation and induction of apoptosis.
